(19) United States
(12) Patent Application Publication
(10) Pub. No.: US 2011/0046661 A1
(43) Pub. Date: Feb. 24, 2011

(54) SURGICAL INSTRUMENT WHICH CAN BE DISASSEMBLED

(75) Inventor: Matthias Kuehn, Elzach (DE)

Correspondence Address: COLLARD & ROE, P.C., 1077 NORTHERN BOULEVARD, ROSLYN, NY, 11576, US

(73) Assignee: Waldemar Link GmbH & Co. KG, Hamburg (DE)

(21) Appl. No.: 12/735,663

(22) PCT Filed: Feb. 6, 2009
(86) PCT No.: PCT/IB2009/000224

§ 371 Date: Nov. 2, 2010

(30) Foreign Application Priority Data
Feb. 6, 2008 (DE) 20 2008 001 675.4

(51) Int. Cl. A61B 17/28

(52) U.S. Cl. 606/206

(57) ABSTRACT

The invention relates to a surgical instrument (1) consisting of a basic element (2), which consists of a first handle part (3) and of a guiding element (4) connected to the handle part, wherein the guiding element extends axially from the handle element, and of a further handle part (5), which is disposed pivotably on the basic element, a sliding element (9f) with a proximal (p) and a distal (d) end, sliding back and forth on the guiding element along the axis thereof, wherein the sliding element is connected at its proximal end to a further handle part by means of a coupling element (11) and the coupling element comprises a driving part (12), which moves a connecting part (13) that corresponds to the driving part and is disposed on the sliding element, back and forth corresponding to the pivoting motion of the further handle part, as well as a functional element (10), which is coupled to the sliding element at the distal end. According to the invention, the following additional distinguishing features are provided: the further handle part (5) is disposed on the basic element (2) so that it cannot be lost, limiting means (16) are provided, which are formed at the proximal end (p) of the first handle part (3), the limiting means (16) are constructed in the form of sliders and are disposed displaceably in such a manner, that they limit the pivoting motion (arrow direction 7, 7′) of the further handle part (5).

The invention relates to a surgical instrument consisting of a base element that consists of a first handle part and a guiding element connected with the handle part, whereby the guiding element extends axially from the handle element, as well as a further handle part that is disposed on the base element. Furthermore, a sliding element that slides back and forth on the guiding element, in its axial expanse, and has a proximal and a distal end, is provided, whereby the sliding element is connected with the further handle part at its proximal end, by way of a coupling element. Furthermore, the coupling element has a driving part that moves a connecting element that corresponds with the driving part and is disposed on the sliding element back and forth, corresponding pivoting movements of the further handle part, as well as a functional element that is coupled with the sliding element at the distal end. The functional element is coupled with the sliding element in non-detachable manner, and is disposed on the guiding element so as to rotate.

### DEFINITION

Instruments of this type are also classified in the group of sliding element instruments. Such sliding element instruments are particularly used in the sector of minimally invasive endoscopic surgery. In general, they have a thin, long shaft that has a diameter between 5 mm and 15 mm. At its distal end, a work tool is provided as a functional element. This work tool generally has a movable tool element that is particularly configured as a gripping or cutting device. The movement of this tool element is brought about by means of the activation device provided at the proximal end of the shaft. In general, this is a scissors-handle device with a fixed branch disposed laterally, and handle branches or handle parts mounted to pivot on the former.

A force transfer element is disposed between the work tool and the handle branch. This serves to exert the force applied by the handle branch to the tool element.

In the following description, the terms “proximal” and “distal” are used for the surgical instrument, to indicate position. The term “proximal” relates to the region of the instrument that is situated away from the patient, whereas the term “distal” refers to the region that is assigned to the patient. This means that the instrument is held and guided by a user in the proximal region.

### STATE OF THE ART

A plurality of surgical instruments that are configured as shaft instruments or sliding element instruments are known from the state of the art.

Thus, for example, a surgical sliding element instrument is known from DE 29815846 U (OLYMPUS WINTER) Sep. 3, 1998, which consists of a fixed handle branch, at the distal end of which a tool element is disposed. The tool element is configured in the form of scissors or forceps, and is operated by way of a ball joint disposed at the proximal end of the sliding element instrument. A cable pull that is disposed in the shaft serves to transfer force.

Furthermore, an instrument for surgical purposes is known from DE 4341734 C (AESCULAP AG) Dec. 8, 1993. This also consists of a fixed handle branch and a further handle branch mounted so as to pivot relative to the former, whereby the further handle branch transfers the applied force to the tool element disposed at the distal end, by way of a force transfer means. The tool element itself is configured as scissors or forceps. In addition, the surgical instrument described here serves to apply a corresponding electrical voltage to the distal end of the tool element.

A surgical punching instrument is known from DE 9307621 U (AESCULAP AG) May 19, 1993. This surgical punching instrument again has a fixed handle branch on which a movable further handle branch is disposed, mounted so as to pivot. The movable handle branch is in turn connected with a sliding element, by way of a coupling element, whereby the sliding element moves back and forth on a guiding element that extends axially in the longitudinal direction of the surgical instrument. The tool element in the form of a punch is disposed on the distal end of the sliding element, as a functional element. The movable handle branch has a driving part that interacts with a connecting part that is disposed in the sliding element. For cleaning reasons, the sliding element can be completely removed from the guiding element. The handle branch that is mounted so as to pivot is disposed on the base element, which also comprises the further handle branch, in non-detachable manner.

A surgical instrument that also has a scissors handle is also known from DE 29922271 (WEINMANN GMBH) Dec. 17, 1999. The scissors handle is formed from a fixed handle branch that is configured as part of the base element, on which a sliding element also slides, and from a further movable handle branch that moves relative to the first handle branch. A tool element in the form of a bone punch is disposed at the free, distal end.

A surgical instrument in the configuration of a sliding element instrument is also configured from U.S. Pat. No. 6,126,674 A (PETER JANZEN) Oct. 28, 1998. The first handle branch is firmly connected with the base element, on of which a sliding element glides. The sliding element in turn is connected with a functional element, or, by way of a connecting element, with a further movable handle branch on the base element.

A surgical instrument that is particularly suitable for endoscopic surgery is represented from U.S. Pat. No. 5,851,214 A (SCOTT W. LARSEN, CHRISTOPHER MCDONNEL, SCOTT W. REED) Sep. 16, 1996. This surgical instrument, in the configuration of a conchotome, has a base element with which a first handle part is firmly connected. The base element furthermore comprises a guiding element that extends axially away from the first handle part. Furthermore, a further handle part is provided, which is movably disposed on the base element. A sliding element that has a proximal and a distal end, in each instance, is disposed on the guiding element itself, sliding back and forth in the axial expanse. At the proximal end, the sliding element is connected with the further handle part by way of a coupling element. The coupling element itself comprises a driving part, whereby a connecting part is disposed on the sliding element, which part has the property of transferring the pivot movement or force exerted by the further handle part to the sliding element.

A surgical instrument for endoscopic use is also known from DE 10 2004 009 200 A1 (Larl Storz GmbH & Co. KG) Feb. 25, 2004. A functional element in the configuration of a cutting or clamping mechanism is known at the distal end of the instrument, which mechanism interacts by way of a sliding element, which in turn is coupled with a handle branch. The sliding element is coupled with the functional element in non-detachable manner, whereby the proximal part can be uncoupled from the handle branch, in order to guarantee cleaning.

A surgical sliding element instrument is known from U.S. Pat. No. 5,507,772 (Shutt et al) May 20, 1994, which has a functional element, for example in the configuration of scissors, at its distal end. This functional element in turn is coupled with a sliding element that interacts with a scissors-like handle branch. By moving the scissors-like handle branch mechanism, the functional element can be opened and closed. Since the instrument is particularly intended for endoscopic use, cleaning is necessary. In order to provide for cleaning of the region of the sliding element, the latter can be removed, in such a manner that the distal part continues to remain connected with the functional element, in articulated manner, whereas the proximal end can be uncoupled from the handle branch by means of activating an unlocking mechanism. Thus, correlation with precise fit, particularly during cleaning procedures of multiple such surgical sliding element instruments, is guaranteed.

DE 199 15 427 A1 (Karl Storz GmbH & Co. KG) Apr. 6, 1999, also shows a medical instrument in the configuration of a sliding element instrument. It differs essentially from the configuration shown in U.S. Pat. No. 5,507,772 in that the uncoupling mechanism of the sliding element is released from the handle branch in different manner. Coupling of the sliding element takes place by way of a driving element of the first handle branch, which is movably disposed on the base body of the instrument. In a specific position, the sliding element can be taken out of this driving element. In order to prevent release from occurring during use of the instrument, a securing element is provided, which restricts the displaceability of the sliding element. The securing element is configured to be U-shaped in cross-section, and can be flipped over the uncoupling region of the sliding element, to secure it. Additional engagement pins secure unintentional opening of the securing element.

### DISADVANTAGES OF THE STATE OF THE ART

Re-usability of surgical instruments using suitable sterilization methods is important. Re-usability is equated with economic efficiency. The instruments contaminated by operations must be sterilized after use, in order to avoid hazards to human life caused by microbiological or non-microbiological residues. However, medical surgical instruments are known which yield insufficient results despite a recognized sterilization method, because of their mechanical structure or function. These are, in particular, such devices that, although they function perfectly in mechanical terms, cannot be sufficiently disassembled so that guiding surfaces, for example, are free of bacteria or the like.

Conchotomes, in particular, have the property that the sliding element cannot be separated from the guiding element, since otherwise their function is no longer guaranteed. Microorganisms embed themselves between the sliding element and the guiding element, and these then lead to the result that the desired degree of sterilization of the instrument is not achieved.

On the other hand, if the sliding element is removed, there is the risk that the same sliding element is not directly set back onto the same guiding surface, since in general, multiple conchotomes are used during operations, and therefore are also sterilized.

Once the sliding element has been fixed in place in the work region, it can pivot about this region. Thus, it is possible to clean the regions of the sliding element without losing the corresponding correlation with the conchotome. In order to prevent the sliding element from unintentionally coming loose from the remainder of the instrument, securing elements are provided. These have the disadvantage, however, that they have a very complex structure, consist of very many parts, and generally must be operated with both hands.

### Task of the Invention

It is the task of the invention to further develop a surgical instrument, for example a conchotome, in terms of its design, in such a manner that at least one disadvantage of the state of the art is avoided.

### Solution for the Task

The basic idea of the solution for the task is that of exposing the problematical geometries, such as gaps, sliding surfaces, and threads, to the sterilization process, and nevertheless guaranteeing the functionality of the instrument after the sterilization process.

Functionality is understood to mean the property that the components that have been coordinated with one another during the production process can be joined together again, without problems, so that the desired property of the functional element, for example of the forceps or the scissors, is maintained.

The solution is proposed in accordance with the characterizing features of claim 1.

### Advantages of the Invention

The significant advantage of the invention is that the problematical functional surfaces of the surgical instrument known from the state of the art now are exposed for the sterilization process by means of a design change, but nevertheless, functionality remains guaranteed. Furthermore, there is the certainty that the sliding element will not unintentionally come loose from the instrument.

The sliding element used in the surgical instrument remains connected with the base element of the surgical instrument in non-detachable manner, in that it remains connected with the functional element of the surgical element in non-detachable manner. In order to expose the guiding surfaces between the sliding element and the guiding element, the sliding element is advantageously removed from the coupling element in a specific position of the further handle part, and flipped away. Because of the articulation onto the functional element, however, the functionality is nevertheless maintained, since the sliding element, which is precisely adapted to the surgical instrument, particularly with regard to its precise fit, remains attached to the same surgical instrument, in non-detachable manner.

So that unintentional removal of the sliding element is not possible, a restriction means, preferably configured as a slide, is disposed on the proximal side on the first handle part, which is an integral part of the base element. The slide is configured in such a manner that it restricts the sliding movement of the sliding element in the proximal direction. In this way, the result is achieved that the opening angle between the handle parts is also restricted.

The opening angles of the two handle parts are divided essentially into two regions, namely a first region in which the instrument is used, and a second region that allows removal of the sliding element. The opening angle of the second region is therefore greater than the opening angle of the first region.

The restriction means is a component that is an integral part of the instrument, and therefore is connected with it in non-detachable manner. It is disposed on the proximal face side of the instrument, and can be operated very easily with a finger, for example a thumb. In order to operate it, it is not necessary to depart from the usual holding position. Furthermore, the surface, which is preferably grooved, can additionally be used to guide the instrument more precisely and to exert pressure in the distal direction with the thumb.

When using the instrument, this restriction means is disposed in such a manner, in a first embodiment, that it restricts the sliding path of the sliding element in the proximal direction, in that the restriction means makes contact with the sliding element. In another exemplary embodiment, the opening angle of the movable handle part is restricted (therefore, indirectly, also the sliding path of the sliding element). Preferably, this happens in the region of the coupling element of the handle, which element interacts with the sliding element. The reason for this is that the forces that act on the restriction means are small. A further advantage consists in that the blocking mechanism is not evident to the user.

The coupling element, which is disposed on the further handle part and comprises a driving part, which corresponds with a connecting part on the sliding element, is designed in such a manner that uncoupling is not possible during use of the instrument. Only by means of active activation of the slide does greater deflection of the handle part become possible, up to the point at which the driving part and the connecting part can be separated from one another.

As a result, it is possible to lift the sliding element out of the driving part and to pivot it about the articulation point on the functional part. The driving part preferably has a slit guide into which a pin that is coupled with the sliding element engages. The pin extends transversely, preferably perpendicular to the longitudinal expanse of the sliding element. In this way, removal from the slit guide is only possible once the handle part is in a position that is unusual for use, and thus the slit guide is perpendicular to the longitudinal expanse of the sliding element. The guiding surfaces that are present between the guiding element and the sliding element are therefore exposed and can be cleaned without any problems.

Preferably, the slide is spring-loaded. This means and also brings with it the advantage that unintentional loosening of the sliding element is essentially not possible. Only by means of active activation, in other words by displacing the slide counter to a spring force, can the sliding element be displaced further in the proximal direction, so that removal from the driving part is then possible. Thus, the user can rely on the fact that when the instrument is activated, unintentional loosening of the sliding element will not occur. Instead, it is provided that when the sliding element is not correctly coupled with the driving part, coupling is necessarily brought about by activation of the instrument.

A further advantageous embodiment provides that the slide does not engage directly at the proximal end of the sliding element, but rather exerts its restriction within the sliding element. This brings with it the advantage that an additional pressure surface is made available for activation of the sliding element, particularly for the thumb, which surface further improves work with the surgical instrument.

In order to avoid unintentional lifting of the sliding element from the guiding element or its guiding surface, guiding means are provided on the side of the sliding element that faces the guiding surface. These guiding means are configured in such a manner that they are preferably T-shaped in cross-section, and engage into an undercut provided in the guiding surface. In this way, lift-off perpendicular to the longitudinal expanse of the sliding element is avoided.

Exclusively in a specific position, namely in which the sliding element is moved beyond its restriction on the proximal side, the recesses in the guiding surface that correspond with the guiding means are also configured in such a manner that no undercut is provided any longer. Therefore it is possible to remove the sliding element from the guiding surface. As a result, double security is present, namely the coupling with the driving part and the swallowtail-like guidance of the sliding element in the guiding means.

Further advantageous embodiments are evident from the following description, the drawings, and the claims.

### DRAWINGS

These show:

FIG. 1 a side view of a surgical instrument according to the invention, namely a conchotome, in the closed state;

FIG. 2 a side view of a surgical instrument according to the invention, namely a conchotome, with the functional element open;

FIG. 3 a perspective view of the surgical instrument shown in FIGS. 1 and 2, but with the sliding element already removed;

FIG. 4 a section through a part of the surgical instrument according to FIG. 3;

FIG. 5 a partial detail of a longitudinal section through the surgical instrument according to FIG. 1.

### DESCRIPTION OF AN EXEMPLARY EMBODIMENT

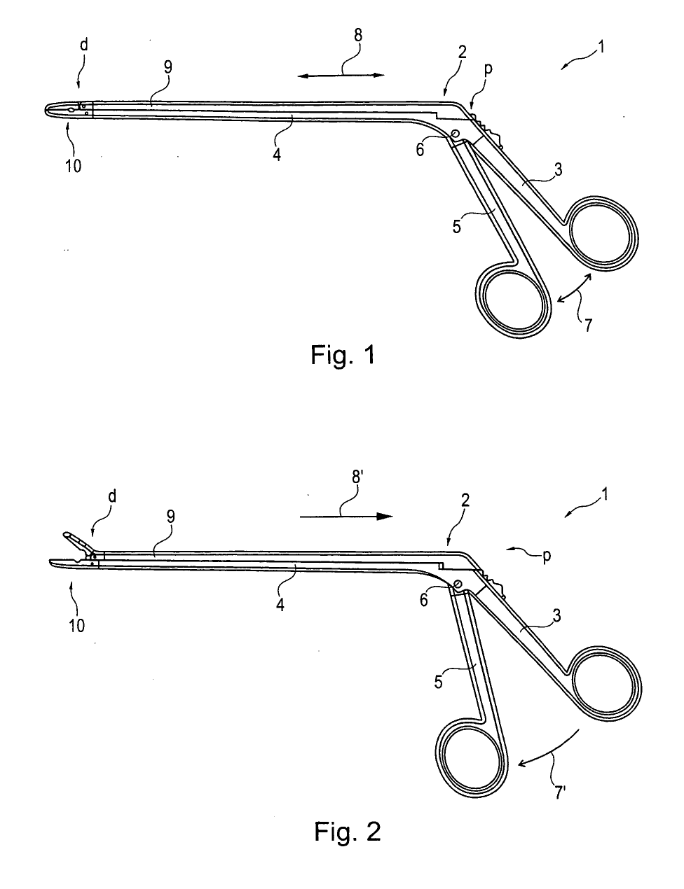

In FIG. 1, a side view of the surgical instrument 1 according to the invention is shown. The surgical instrument 1 consists essentially of a base element 2. The base element 2 in turn consists of a first handle part 3 and a guiding element 4 connected on the handle part 3. The guiding element 4 extends axially away from the one free end of the handle part 3. Guiding element 4 and handle part 3 are connected with one another in one piece. Furthermore, a further handle part 5 is provided, which can be pivoted on the base element 2. In order to achieve pivotability, a bearing element 6 is provided, which makes it possible to pivot the further handle part 5 relative to the first handle part 3, in and opposite the arrow direction 7.

Preferably, the further handle part 5 is disposed on the base element 2 in non-detachable manner (but so that it can rotate). A sliding element 9 is disposed on the guiding element 4, so as to be displaceable in and opposite the arrow direction 8. This sliding element 9 has a proximal end p and a distal end d. A functional element 10 is provided at the distal end d of the sliding element 9, which functional element is coupled both with the base element 2, particularly with the guiding element 4, and with the sliding element 9. In the exemplary embodiment shown here, the surgical instrument 1 represents a conchotome, so that the distal end d of the surgical instrument 1 provides a cutting device as the tool means.

In FIG. 2, the further handle part 5 is pivoted relative to the first handle part 3, in the arrow direction 7′. As a result, the sliding element 9 is moved in the arrow direction 8′, and the functional element 10 opens. From this, it is evident that the further handle part 5 is coupled with the sliding element 9.

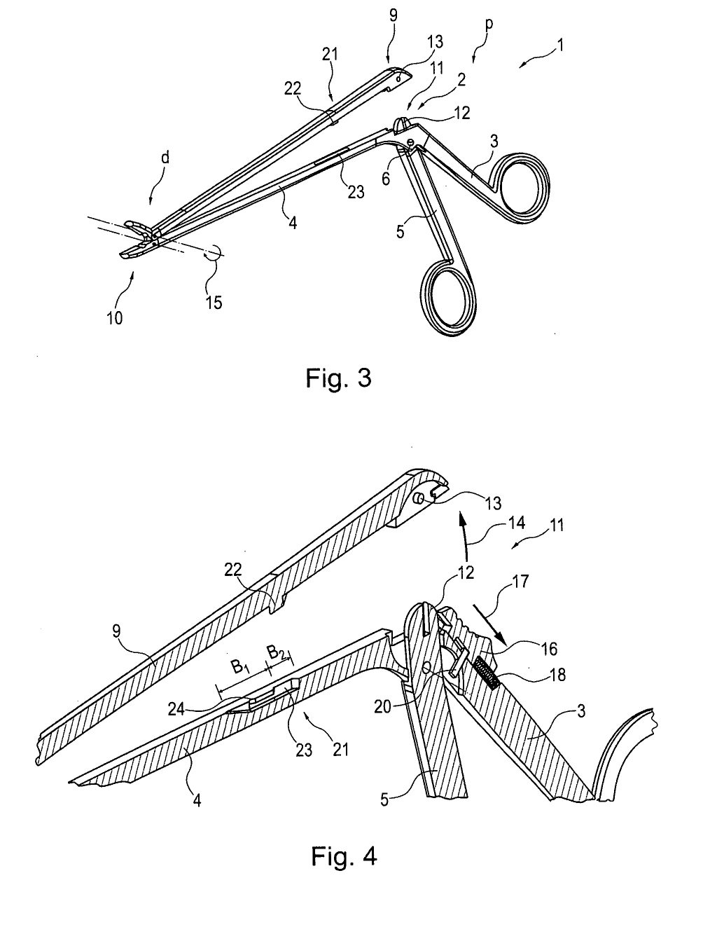

In FIG. 3, it is shown how the sliding element 9 can be removed from the base element 2. For this purpose, the further handle part 5 is in a special position, in which the sliding element 9 can be removed from the coupling element 11 and from a connecting part 13 that corresponds with the driving part 12.

In FIG. 4, an enlarged representation is shown, particularly of the coupling element 11. The driving part 12 is connected in one piece with the further handle part 5, and has a U-shaped recess at its free end. In the position shown in FIG. 4, the U-shaped recess is practically perpendicular to the longitudinal expanse of the guiding element 4 or of the sliding element 9 mounted on the guiding element 4. The sliding element 9 has already been removed from the U-shaped recess of the coupling element 11, particularly of the driving part 12, in the arrow direction 14, whereby the distal end d of the sliding element 9 continues to be coupled with the functional element 4, and in order to pivot the latter, as shown in FIG. 3, in or opposite the arrow direction 15, with rotational mobility, at least in restricted manner. In this way, it is possible to clean the guiding surface between sliding element 9 and guiding element 4, without completely removing the sliding element 9, which is adapted to the base element 2.

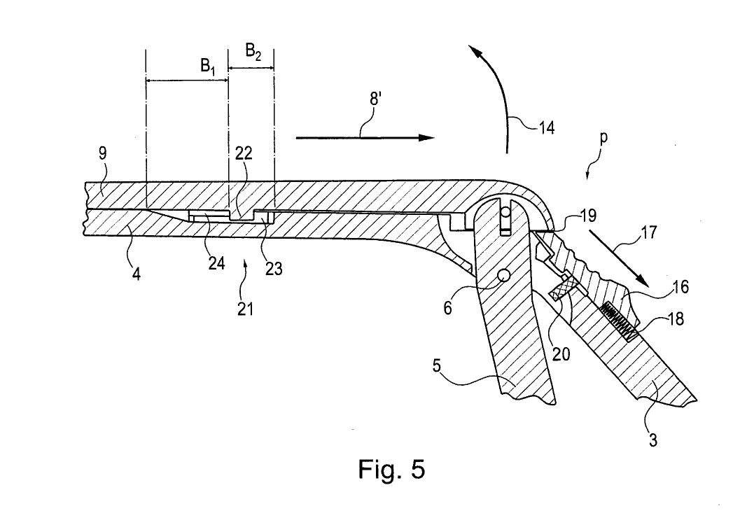

In order to lift the sliding element 9, as shown in FIGS. 3 and 4, from the guiding element 4 in the arrow direction 14 in the first place, the further handle part 5, as has already been explained, as well, must be brought into a defined position that is practically perpendicular to the longitudinal expanse of the guiding element 4 (FIG. 5). However, this position cannot easily be reached, since it is supposed to be prevented that when the surgical instrument 1 is in use, the sliding element 9 should not unintentionally come loose from the guiding element 4. In order to achieve this, a restriction means 16 is provided, which restricts the movement of the sliding element 9 in the arrow direction 8′. In order to release movement in the arrow direction 8′, it is necessary that the restriction means 16 is displaced in arrow direction 17, against the force of a spring 18, until the free end 19 of the restriction means 16 releases the proximal end p of the sliding element 9.

When the restriction means 16 takes on the function that the sliding element 9 is not displaced further than the position shown in FIG. 5, then the free end 19 of the restriction means 16 is disposed in the region of the sliding element 9, and limits the pivoting movement of the further handle part 5 and/or that of the sliding element 9, for one thing.

Alternative embodiments for restricting the movement of the sliding element 9 in the arrow direction 8′ are numerous and varied. For example, it is also possible that not the sliding element 9, but rather the further handle part 5 is restricted in its deflection. A further alternative provides that an additional element, which is disposed on the guiding element 4 of the base element 2, restricts the movement and releases it for further displacement and then for removal of the sliding element 9, as required, by means of activation of an additional slide or pivoting mechanism.

The restriction means 16 described in the figures is additionally restricted with its movement device by means of a bolt element 20.

In order to prevent the sliding element 9 from unintentionally coming loose from the guiding element 4, the guiding means 21 shown in FIGS. 3 to 5 are provided. Such a guiding means 21 consists of a first guiding part 22, disposed on the sliding element 9 on the side facing the guiding element 4, which part is preferably configured to be T-shaped in cross-section. This first guiding part 22 corresponds with a further guiding part 23, which is disposed on the side of the guiding element 4, facing toward the sliding element 9. The further guiding part 23 is a recess that has an undercut 24 in a defined region B1, which undercut interacts with the T-shaped configuration of the first guiding part 22. Furthermore, a further region B2 is provided, which does not have an undercut 24 and is dimensioned in such a manner that the first guiding part 22 can be completely removed from the further guiding part 23. This also corresponds to the position in which the connecting part 13 of the sliding element 9 can be removed from the U-shaped configuration of the driving part 12.

In order to join the sliding element 9 together with the base element 2, all that is necessary is to ensure that the second handle part 5 or its U-shaped driving part 12 is oriented practically perpendicular to the longitudinal expanse of the guiding element 4 (as shown in FIG. 5), so that the connecting part 13 of the sliding element 9 can be pressed into the U-shaped recess. At the same time, and completely independently, the first guiding part 22 is introduced into the region B2 of the second guiding part 23, and thus prepares for the non-detachable connection between the sliding element 9 and the guiding element 4. The back and forth movement of the sliding element 9 is achieved by means of moving the second handle part 5 relative to the first handle part 3, and, at the same time, the restriction means 16 automatically goes into effect.

When the surgical instrument 1 has been in use, all that is necessary is to activate the restriction means 16 in the arrow direction 17. To open the second handle part 5 wide relative to the first handle part 3, in the arrow direction 7′ (FIG. 2), and to press the sliding element 9 in the arrow direction 14 (FIG. 5) with the thumb. As a result, the position shown in FIG. 3 can be achieved, so that the guiding surfaces that form between the sliding element 9 and the guiding element 4 can also be cleaned properly by means of the sterilization process.

The restriction means 16 is designed in such a manner that it engages on the underside of the sliding element 9. The outer restriction then serves as a stop. Another embodiment provides that the restriction means 16 engages on the proximal side p of the sliding element 9, as a stop element. Fundamentally, the restriction element 16 serves to restrict the displacement path of the sliding element 9, independent of the region in which it is disposed, in such a manner that the driving part 12 does not get into the perpendicular position for removing the connecting part 13 and thus the sliding element 9.

### REFERENCE SYMBOL LIST

1 surgical instrument

2 base element

3 handle part

4 guiding element

5 further handle part

6 bearing element

7 arrow direction

7′ arrow direction

8 arrow direction

8′ arrow direction

9 sliding element

10 functional element

11 coupling element

12 driving part

13 connecting part

14 arrow direction

15 arrow direction

16 restriction means

17 arrow direction

18 spring

19 free end

20 bolt element

21 guiding means

22 first guiding part

23 second guiding part

24 undercut

p proximal end

d distal end

B1 region

B2 region

1. Surgical instrument, consisting of
a base element that consists of a first handle part and a guiding element connected with the handle part, whereby the guiding element extends axially from the handle element, as well as
a further handle part that is disposed on the base element so as to pivot,
a sliding element that slides back and forth on the guiding element, in its axial expanse, and has a proximal and a distal end, whereby the sliding element is connected with the further handle part at its proximal end, by way of a coupling element,
the coupling element comprises a driving part that moves a connecting part that corresponds with the driving part and is disposed on the sliding element back and forth, corresponding to pivoting movements of the further handle part, as well as
a functional element that is coupled with the sliding element at the distal end,
the functional element is coupled with the sliding element in non-detachable manner, and with the guiding element so as to rotate,
wherein
the further handle part (5) is disposed on the base element (2) in non-detachable manner,
a restriction means (16) is provided, which is disposed at the proximal end (p) of the first handle part (3),
the restriction means (16) is configured in scissors-like manner, and
this means is disposed in displaceable manner, in such a manner that it restricts the pivoting movement (arrow direction 7, 7′) of the further handle part (5).

2. Surgical instrument according to claim 1, wherein the restriction means (16) is spring-loaded with a spring (18), in such a manner that it automatically moves into its locking position.

3. Surgical instrument according to claim 2, wherein the spring force of the spring (18) acts on the restricting means (16) in the direction of the sliding element (4).

4. Surgical instrument according to claim 1, wherein the restriction means (16) acts below or within the sliding element (9).

5. Surgical instrument according to claim 1, wherein a guiding means (21) is provided between the proximal end (p) and the distal end (d) of the sliding element (9), which means corresponds with the guiding element (4).

6. Surgical instrument according to claim 5, wherein a guiding surface is provided between the sliding element (9) and the guiding element (4), whereby at least one T-shaped first guiding part (22) extends away from the sliding element (9), which part interacts with a further second guiding part (23) provided in the guiding surface of the guiding element (4).

7. Surgical instrument according to claim 6, wherein the second guiding part (23) is configured in such a manner that the T-shaped first guiding part (22) undercuts a defined region (B1) over this region (B1), and wherein a further region (B2) is provided starting from a defined position of the further handle part (5), relative to the first handle part (3), in which region the first guiding part (22) can be removed.
